(19) United States
(12) Patent Application Publication
(10) Pub. No.: US 2017/0042591 A9
(43) Pub. Date: Feb. 16, 2017

(54) INTRAMEDULLARY ANCHOR-SCREW FRACTURE FIXATION

(71) Applicant: Extremity Designs, LLC, Tampa, FL (US)

(72) Inventors: Mark Allen Mighell, Tampa, FL (US); Sergio Gutierrez, Tampa, FL (US)

(21) Appl. No.: 14/569,420

(22) Filed: Dec. 12, 2014

Related U.S. Application Data
(60) Provisional application No. 61/915,144, filed on Dec. 12, 2013.

(51) Int. Cl. A61B 17/72

(52) U.S. Cl. CPC A61B 17/7233; A61B 17/7266; A61B 17/7258

(57) ABSTRACT

The present invention relates to an improved intramedullary fracture fixation device, the device including a first fastener comprising a head and an at least partially threaded shaft extending from the head, a tiltable load spreading element adapted to couple to the first fastener and having a bone-contacting surface defining bone engagement features and a head-contacting surface, and a second fastener having external threads and a central bore for receiving the threaded shaft of the first fastener. The central bore defines an inner wall surface which is at least partially threaded, the inner wall surface being configured for mating engagement on the outer surface of the shaft of the first fastener such that rotation of the first fastener relative to the second fastener causes the translation of the first fastener relative to the second fastener along the shaft. The load spreading element is configured and dimensioned to interact operatively with at least a surface of the bone and at least a surface of the head to spread the load exerted by the head on the bone over a large bone surface area and, optionally, to prevent the head from completely passing through the bone. The present invention permits a minimally invasive repair of bone fractures that may otherwise be treated through larger incisions. The present invention also relates to the method of fixation of the device.

### FIELD OF INVENTION

This invention relates to intramedullary fracture fixation. More specifically, this invention relates to a multi-piece anchor-screw construct intended for intramedullary fixation of long bone fractures.

### BACKGROUND OF THE INVENTION

Severe bone fractures are most commonly repaired with open reduction and internal fixation using plates and screws attached to the cortex of the bone. The preferred method to gain access to the fracture site is by making a large incision through the skin and muscles. Once the fracture has been exposed, the plate is then attached to the bone fragment(s) using screws. The placement of the bone plate will aid in healing by minimizing the amount of interfragmentary movement. Once the plate is in place, the wound is sutured closed, leaving behind a large scar.

Intramedullary anchor-screw fixation gives the surgeon an alternative to plate fixation by creating a minimally invasive procedure with better aesthetic, as well as functional outcomes. By utilizing the intramedullary canal for the location of the anchor-screw system, it will provide for a lower profile fixation, minimizing problems of prominent hardware, as well as providing improved mechanical resistance to forces across the fixation-bone interfaces.

### SUMMARY OF INVENTION

This new invention in fracture fixation allows for minimally invasive treatment with minimal scarring. It is therefore an object of this invention to provide minimally invasive fracture fixation in the diaphysis of long bones, including: the ulna, clavicle, humerus, radius, carpals, metacarpals, phalanges, femur, tibia, fibula, tarsals and metatarsals.

In accord with these objects, an intramedullary anchor-screw fixation system is provided. The system includes an inner and outer threaded nut and a screw that attaches to said nut. The screw has a multi-axial head to be able to accommodate irregular bony geometry. The nut and screw can be made from different materials, such as, but not limited to: titanium, titanium alloys, stainless steel, tantalum and bioabsorbable materials.

To position the current invention into the fractured bone, the surgeon would follow the following procedure: An incision would be made at one side of the fracture. A drill bit would then be used to drill through both fragments. The bi-threaded nut would be screwed through the first fragment and then on to the second fragment. The screw would then be used to anchor the first fragment onto the second fragment by locking into the bi-threaded nut. The two fragments would be compressed against each other to prevent torsional instability. The wound is then sutured closed, creating minimal scarring.

In accord with another embodiment, the anchor-screw system is made in differing widths, lengths and shapes depending on both the type of fracture and the long bone being repaired.

### BRIEF DESCRIPTION OF THE DRAWINGS

FIG. 1 is a perspective view of the anchor-screw device implanted in an ulna.

FIG. 2 is a perspective view of the anchor-screw device implanted in the proximal ulna.

FIG. 3 is a proximal view (i.e., elbow) of the screw and spiked washer implanted in the ulna.

FIG. 4 is a side view of the anchor-screw device implanted in an ulna.

FIG. 5 is a perspective view of the anchor-screw device.

FIG. 6 is an anterior view of the anchor-screw device implanted in a clavicle.

FIG. 7 is a close-up anterior view of the anchor-screw device implanted in a clavicle.

### DETAILED DESCRIPTION OF THE PREFERRED EMBODIMENT

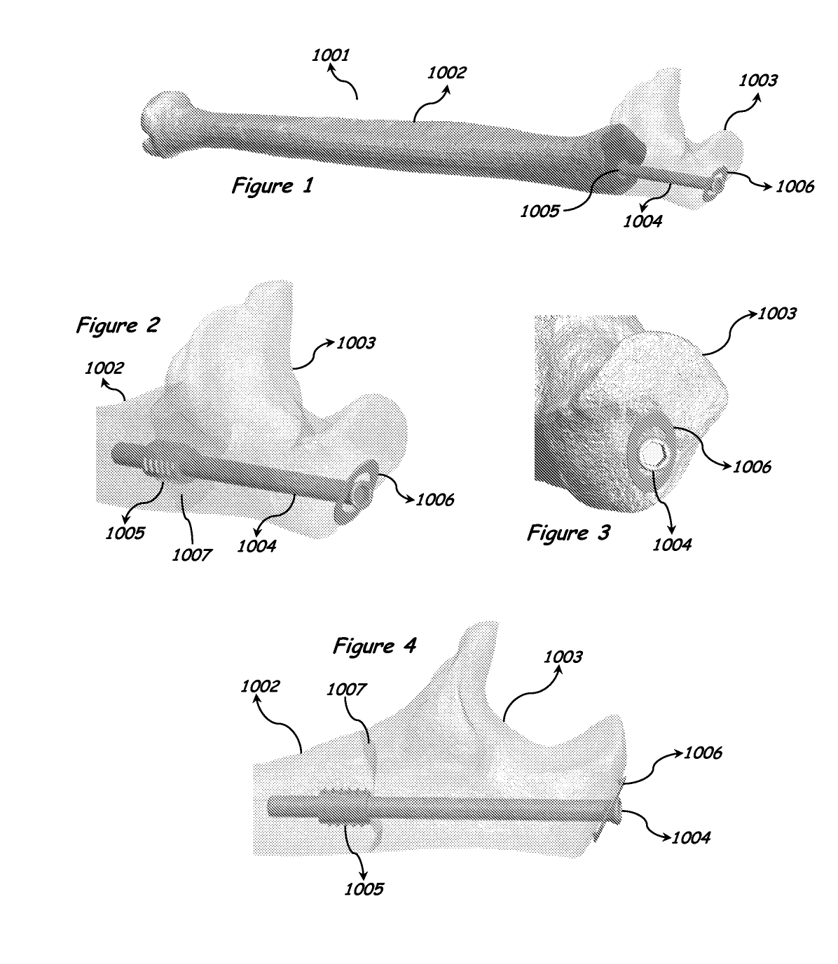

The preferred embodiment of the present invention is shown in FIGS. 1-7.

As shown in FIG. 1, a perspective view of the ulna 1001 showing a distal fracture fragment 1002 and a proximal fracture fragment 1003. The anchor-screw device consists of: a distal threaded nut 1005 that is screwed into the distal fragment 1002, a proximal screw 1004 that is implanted through a hole in the proximal fragment 1003, and a spiked washer 1006 that is inserted between the proximal fragment 1003 and the proximal screw 1004.

As shown in FIG. 2, a perspective view of the elbow showing the proximal fragment 1003, the distal fragment 1002 and the fracture site 1007, the threaded nut 1005, the proximal screw 1004 and the spiked washer 1006.

As shown in FIG. 3, a close-up view of the elbow showing the proximal fragment 1003, the proximal screw 1004 and the spiked washer 1006.

As shown in FIG. 4, a side view of the elbow showing the proximal fragment 1003, the distal fragment 1002 and the fracture site 1007, the threaded nut 1005, the proximal screw 1004 and the spiked washer 1006.

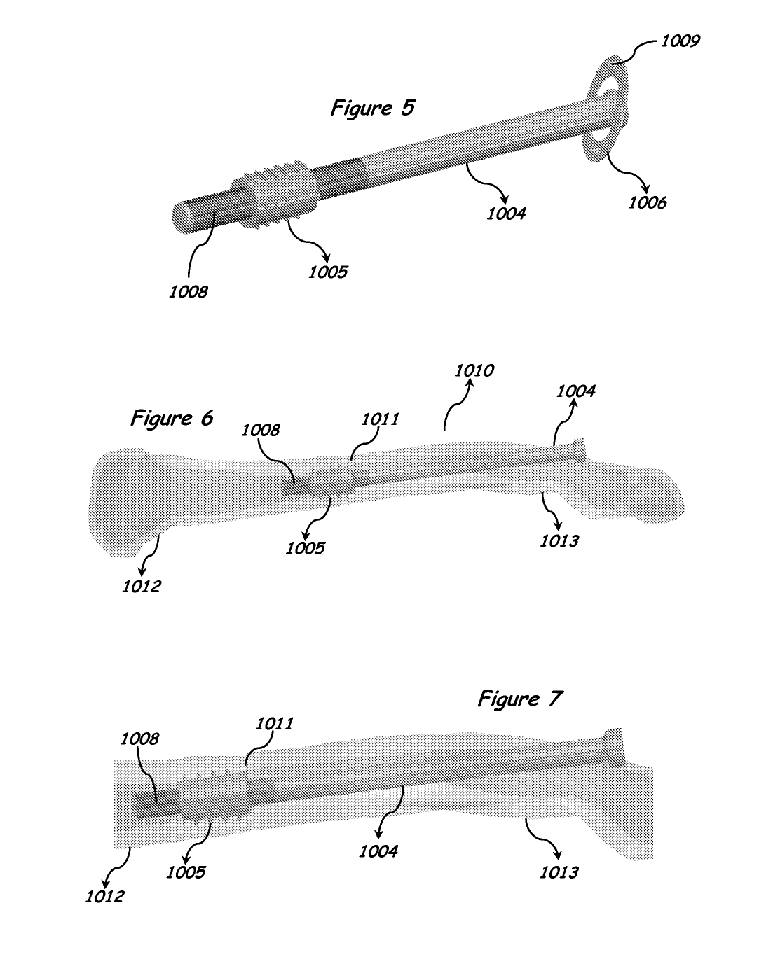

As shown in FIG. 5, a perspective view of the anchor-screw device showing the threaded nut 1005, the proximal screw 1004 with threads 1008 and the spiked washer 1006 showing the spikes 1009.

Turning now to FIGS. 6 and 7, an anterior view of the present embodiment implanted in a transverse fractured (1011) clavicle 1010. The anchor-screw device is implanted by threading the threaded screw 1005 into the proximal clavicle fragment 1012, and then inserting the threaded screw 1004 through the distal clavicle fragment 1013.

The principles, preferred embodiments and modes of operation of the present invention have been made apparent in the foregoing description.

The invention should not be construed as limited to the described embodiments. Instead, the embodiments described herein should be regarded as illustrative and not restrictive in any way. Variations and changes may be made by others without departing from the intent of the present invention as defined by the following claims:

What is claimed is:

1. An intramedullary fixation system for repairing a bone, comprising:
a first fastener comprises a head and an elongated body defining a proximal portion and a distal portion with an outer surface which is at least partially threaded;
a load spreading element adapted to couple to the first fastener comprises a bone contacting surface and a head-contacting surface; and
a second fastener comprises an outer surface having one or more bone engagement features and a longitudinal central bore for receiving the threaded distal portion of the first fastener, the central bore defines an inner wall surface which is at least partially threaded, the inner wall surface being configured for mating engagement on the outer surface of the distal portion of the first fastener such that rotation of the first fastener relative to the second fastener causes the translation of the first fastener relative to the second fastener along the elongated body,
wherein the load spreading element is tiltable to substantially conform to a surface of the bone.

2. The intramedullary fixation system of claim 1, wherein the load spreading element is a washer having one or more bone engagement features on the bone-contacting surface.

3. The intramedullary fixation system of claim 2, wherein the proximal portion of the elongated body defines a first cross-sectional area and a second cross-sectional area proximate a distal surface of the head, and the second cross-sectional area is smaller than the first cross-sectional area.

4. The intramedullary fixation system of claim 2, wherein the washer has a thickness of about 1-5 mm.

5. The intramedullary fixation system of claim 2, wherein the bone engagement features of the second fastener and the washer are spikes, hooks, threads, pegs or a combination thereof.

6. The intramedullary fixation system of claim 2, wherein the head comprises a proximal surface defining a driver engagement feature and a distal surface being flat, tapered, partially spherical or hemispherical.

7. The intramedullary fixation system of claim 6, wherein the head has a maximum outer diameter and the washer has a minimum inner diameter, and the minimum inner diameter is smaller than the maximum outer diameter.

8. The intramedullary fixation system of claim 2, wherein the bone engagement features on the outer surface of the second fastener are configured differently than the threads on the inner wall surface of the second fastener.

9. The intramedullary fixation system of claim 8, wherein the bone engagement features on the outer surface of the second fastener are one of helical threads and circumferentially spaced-apart columns of threads.

10. The intramedullary fixation system of claim 7, wherein the second fastener has a length of about 5-40 mm.

11. The intramedullary fixation system of claim 10, wherein the outer surface of the second fastener has a tubular shape or a conical shape.

12. The intramedullary fixation system of claim 11, wherein the central bore of the second fastener has a diameter of about 3-8 mm.

13. The intramedullary fixation system of claim 12, wherein the threaded portion of the distal portion of the first fastener extends for less than approximately 70% of an entire length of the elongated body of the first fastener.

14. The intramedullary fixation system of claim 13, wherein at least a portion of the proximal portion of the first fastener is smooth and unthreaded.

15. The intramedullary fixation system of claim 2, wherein the first fastener is cannulated.

16. The intramedullary fixation system of claim 2, wherein at least one of the first and second fasteners has a tapered distal portion.

17. The intramedullary fixation system of claim 1, wherein the second fastener is constructed and arranged to flex radially outwards when one of a torque and energy is applied to the second fastener.

18. The intramedullary fixation system of claim 17, wherein the second fastener comprises two or more axially-extending legs.

19. The intramedullary fixation system of claim 1, wherein the load spreading element is removably coupled to the first fastener.

20. An intramedullary fixation system for repairing a bone, comprising:
a first fastener comprises a head and an elongated cylindrical body defining a proximal portion and a distal portion with an outer surface which is at least partially threaded; and
a second fastener comprises an outer surface which is at least partially threaded and a central bore for receiving the threaded distal portion of the first fastener, the central bore defines an inner wall surface which is at least partially threaded, the inner wall surface being configured for mating engagement on the outer surface of the distal portion of the first fastener such that rotation of the first fastener relative to the second fastener causes the translation of the first fastener relative to the second fastener,
wherein the second fastener is constructed and arranged to flex radially outwards when one of a torque and energy is applied to the second fastener.

21. The intramedullary fixation system of claim 20 further comprises a washer adapted to couple to the first fastener, the washer comprises a bone contacting surface defining one or more bone engagement features and a head-contacting surface.

22. The intramedullary fixation system of claim 21, wherein the proximal portion of the elongated cylindrical body defines a first cross-sectional area and a second cross-sectional area proximate the distal surface of the head, and the second cross-sectional area is smaller than the first cross-sectional area.

23. The intramedullary fixation system of claim 20, wherein the second fastener further comprises two or more axially-extending legs.

24. An intramedullary fixation system for repairing a bone, comprising:
a first fastener comprises a head and an elongated cylindrical body defining a proximal portion and a distal portion with an outer surface which is at least partially threaded; and
a second fastener comprises an outer surface which is at least partially threaded and a central bore for receiving the threaded distal portion of the first fastener, the central bore defines an inner wall surface which is at least partially threaded, the inner wall surface being configured for mating engagement on the outer surface of the distal portion of the first fastener such that rotation of the first fastener relative to the second fastener causes the translation of the first fastener relative to the second fastener,
wherein the proximal portion of the elongated cylindrical body defines a first cross-sectional area and a second cross-sectional area proximate a distal surface of the head, and the second cross-sectional area is smaller than the first cross-sectional area.

25. A method of performing and repairing an osteotomy, comprising the steps of:
drilling a hole through an end of a bone and into the medullary canal of the bone substantially along a longitudinal axis of the bone;
(a) sectioning the bone at a desired location;
(b) moving the sectioned proximal bone fragment off to the side;
(c) inserting a second fastener in the hole drilled in the sectioned distal bone fragment to a depth below the site of the osteotomy;
(d) repositioning the sectioned proximal bone fragment to the sectioned distal bone fragment; and
(e) reducing the sectioned bone by inserting a first fastener through the sectioned proximal bone fragment and into the medullary canal of the bone and securing the first fastener to the second fastener.

26. The method of claim 25, wherein the first fastener comprises a head and an elongated cylindrical body defining a proximal portion and a distal portion with an outer surface which is at least partially threaded.

27. The method of claim 26, wherein the second fastener comprises a central bore for receiving the threaded distal portion of the first fastener, the central bore defines an inner wall surface which is one of deformable and at least partially threaded.

28. The method of claim 27, wherein the second fastener comprises an outer surface having one or more bone engagement features.

29. The method of claim 28, wherein a washer is coupled to the first fastener between a surface of the bone and a distal surface of the head.

30. The method of claim 25, wherein the step of repositioning includes anatomically realigning the sectioned proximal portion to the sectioned distal portion.

31. The method of claim 25, wherein the depth below the site of the osteotomy is about 1 cm.

32. The method of claim 25, wherein the bone is an ulna.

33. A method of performing and repairing an osteotomy, comprising the steps of:
drilling a hole through an end of a bone and into the medullary canal of the bone substantially along a longitudinal axis of the bone;
(a) sectioning the bone at a desired location;
(b) moving the sectioned distal bone fragment off to the side;
(c) inserting a second fastener in the hole drilled in the sectioned proximal bone fragment to a depth below the site of the osteotomy;
(d) repositioning the sectioned distal bone fragment to the sectioned proximal bone fragment; and
(e) reducing the sectioned bone by inserting a first fastener through the sectioned distal bone fragment and into the medullary canal of the bone and securing the first fastener to the second fastener.

34. A method of repairing a bone fracture, comprising the steps of:
drilling a hole through an end of a bone and into the medullary canal of the bone substantially along a longitudinal axis of the bone;
(a) moving the proximal bone fragment off to the side;
(b) inserting a second fastener in the hole drilled in the distal bone fragment to a depth below the site of the fracture;
(c) repositioning the proximal bone fragment to the distal bone fragment; and
(d) reducing the fracture by inserting a first fastener through the proximal bone fragment and into the medullary canal of the bone and securing the first fastener to the second fastener.

35. The method of claim 34, wherein the first fastener comprises a head and an elongated cylindrical body defining a proximal portion and a distal portion with an outer surface which is at least partially threaded.

36. The method of claim 35, wherein the second fastener comprises a central bore for receiving the threaded distal portion of the first fastener, the central bore defines an inner wall surface which is one of deformable and at least partially threaded.

37. The method of claim 36, wherein the second fastener comprises an outer surface having one or more bone engagement features.

38. The method of claim 37, wherein a washer is coupled to the first fastener between a surface of the bone and a distal surface of the head.

39. The method of claim 34, wherein the depth below the site of the fracture is about 1 cm.

40. A surgical kit for repairing a bone, comprising:
a first fastener comprises a head and an elongated body having one or more threads on an outer surface;
a load spreading element adapted to couple to the first fastener comprises a bone contacting surface and a head-contacting surface; and
a second fastener comprises an outer surface having one or more bone engagement features and a longitudinal central bore for receiving the threaded portion of the first fastener, the central bore defines an inner wall surface which is at least partially threaded, the inner wall surface being configured for mating engagement on the outer surface of the threaded portion of the first fastener such that rotation of the first fastener relative to the second fastener causes the translation of the first fastener relative to the second fastener along the elongated body.

41. The surgical kit of claim 40, wherein the load spreading element is a washer.
